(19) United States
(12) Patent Application Publication
(10) Pub. No.: US 2006/0282102 A1
(43) Pub. Date: Dec. 14, 2006

(54) Method and apparatus for holding suture ends to facilitate tying of knots

(76) Inventors: Anthony A. Nobles, Fountain Valley, CA (US); Raymond S. Lee, Irvine, CA (US); Steven E. Decker, Anaheim, CA (US); Benjamin G. Brosch, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/438,619

(22) Filed: May 22, 2006

Related U.S. Application Data
(60) Provisional application No. 60/683,701, filed on May 23, 2005.

(51) Int. Cl. A61B 17/04

(52) U.S. Cl. 606/148

(57) ABSTRACT

A knot-tying assist device allows a physician to remotely tie and cinch a knot in two ends of a suture extending from an incision in a vessel or organ of a patient in order to seal an opening in the vessel or organ. The knot-tying assist device includes a first receiving portion and a second receiving portion spaced apart along the device for receiving and holding the two ends of the suture. The receiving portions may hold the suture ends loosely, or alternatively may hold the suture ends under tension to facilitate tying the knot. The knot-tying device further includes an engagement portion to stabilize the knot-tying assist device relative to the treatment location. The engagement portion may assist in maintaining tension on the two suture ends so that the suture can hold the incision closed prior to the knot being tied and cinched.

### CROSS-REFERENCE TO RELATED APPLICATION

This application claims the benefit of U.S. Provisional Application No. 60/683,701, filed on May 23, 2005, the entirety of which is hereby incorporated by reference.

### BACKGROUND OF THE INVENTION

1. Field of the Invention

Certain embodiments of the invention relate to suturing incisions, and more specifically, to the use of sutures for closing incisions in vessels and organs within a body.

2. Description of the Related Art

Surgeons frequently encounter the need to close incisions, wounds, or otherwise join tissue portions with a suture. After passing the suture through the tissue portions, the surgeon must tie and cinch the suture to draw the tissue portions together and prevent them from separating. When sutures are tied in a region having restricted access, such as the end of a tissue tract leading to an artery, the surgeon is presented with special challenges. Sutures can often be difficult to handle, thereby increasing the time that it takes for a surgeon to tie a suture. Often, an assistant may be needed to hold suture ends apart while allowing a surgeon to tie a knot. Accordingly, what is needed is a faster and more effective way to tie and cinch a suture.

### SUMMARY OF THE INVENTION

The embodiments of the present invention describe various methods and apparatus for tying and cinching suture knots. When two ends of a suture extend away from an incision in a vessel or an organ of a patient, the preferred embodiments provide a method and apparatus for desirably separating the two ends of the suture, and optionally, for applying tension to each suture end to hold the suture taut while the suture ends are being tied.

In one embodiment, a knot-tying assist device is provided. The device preferably includes a first receiving portion and a second receiving portion, each receiving portion adapted to hold a portion of a suture. The receiving portions may loosely hold the suture portions, or more firmly engage the suture portions to hold tension on the suture. In one embodiment, the receiving portions are provided as part of a resilient block of material, such as silicone or rubber, with the receiving portions defined by slits cut into the resilient material. In another embodiment, the receiving portions are provided as arms extending away from each other. The arms may have receiving portions to loosely engage the suture ends, or may include a holding mechanism, such as a resilient material such as silicone or rubber, with slits adapted to engage the suture ends.

In one embodiment, the knot-tying assist device also includes a post that may be insertable into an introducer or directly into an incision, or other mechanism to stabilize the device relative to an introducer or an incision. The post may be provided between the two receiving portions, and when inserted into an introducer or into an incision may apply tension on the suture by holding the suture against the inner wall of the introducer or against the incision. When the post is connected to receiving portions that hold tension on the suture portions, tension is advantageously held on the suture by both the engagement of the post with the introducer or incision, as well as by the force on the suture portions being held within the receiving portions. This tension advantageously allows the suture to hold the treatment location closed prior to the knot being tied and cinched.

In another embodiment, the post may be removable from the introducer or incision by providing an ejection mechanism with the device. The ejection mechanism may comprise a plunger that is pressed down relatively to the post. A lower end of the plunger preferably engages an upper end of an introducer, or the skin of a patient or other surface, such that as the plunger is pressed downward, the post can be lifted upward and be removed from the incision or the introducer.

In one embodiment, a method of tying two suture ends extending from a treatment location is provided. The method comprises holding each of two suture portions in a device that separates the two suture portions a desired distance from each other. The sutures can be held either loosely or tightly. A knot is tied with the two suture ends while the two suture portions are held. The suture portions are removed from the device, and the knot is cinched down to the treatment location.

In another embodiment, a method is provided of holding tension on a suture used to tie an opening in the body. The tension may be held by inserting a post into an introducer to hold suture portions against an inner wall of the introducer. Alternatively or additionally, tension may be held by engaging suture portions with suture receiving portions that grip the suture portions to hold tension. In one preferred embodiment, the receiving portions hold each of two suture portions a distance apart from each other to separate the suture portions.

### BRIEF DESCRIPTION OF THE DRAWINGS

FIG. 1 is a perspective view of a wound site having a pair of suture ends extending therefrom.

FIGS. 2A and 2B are perspective views of a first embodiment a knot-tying assist device.

FIGS. 2C and 2D illustrates the device of FIGS. 2A and 2B holding two suture portions while engaging an introducer.

FIG. 3A is a perspective view of a second embodiment of a knot-tying assist device.

FIG. 3B illustrates the device of FIG. 3A inserted into an introducer and holding two suture portions.

FIGS. 4A and 4B are perspective views of a third embodiment of a knot-tying assist device.

FIGS. 5A and 5B are perspective views of a fourth embodiment of a knot-tying assist device.

FIGS. 6A and 6B are perspective views of a fifth embodiment of a knot-tying assist device.

FIG. 7A is a perspective view of a sixth embodiment of a knot-tying assist device.

FIG. 7B illustrates the knot-tying assist device of FIG. 7A inserted into an introducer and holding two suture portions.

FIG. 8 is a perspective view of a seventh embodiment of a knot-tying assist device.

FIG. 9 is a perspective view of an eighth embodiment of a knot-tying assist device.

FIG. 10 is a perspective view of a ninth embodiment of a knot-tying assist device.

FIG. 11 is a perspective view of a tenth embodiment of a knot-tying assist device.

FIG. 12 is a perspective view of an eleventh embodiment of a knot-tying assist device.

FIGS. 13A and 13B are perspective views of a twelfth embodiment of a knot-tying assist device.

FIG. 13C illustrates the device of FIGS. 13A and 13B being prepared to be inserted into an introducer.

FIG. 13D illustrates the device of FIG. 13C inserted into an introducer.

FIG. 14 illustrates a thirteenth embodiment of a knot-tying assist device in use.

FIG. 15 illustrates a knot being cinched using a knot-cinching device.

### DETAILED DESCRIPTION OF THE PREFERRED EMBODIMENTS

The preferred embodiments of the present invention described below relate particularly to tying the ends of a suture into a knot. More particularly, the preferred embodiments relate to tying the ends of a suture extending from a treatment location of a patient. The treatment location may be any desired location, such as an arterial vessel, a venous vessel or any other body tissue. Suture ends may be the ends of the same suture, or may be the ends of separate sutures.

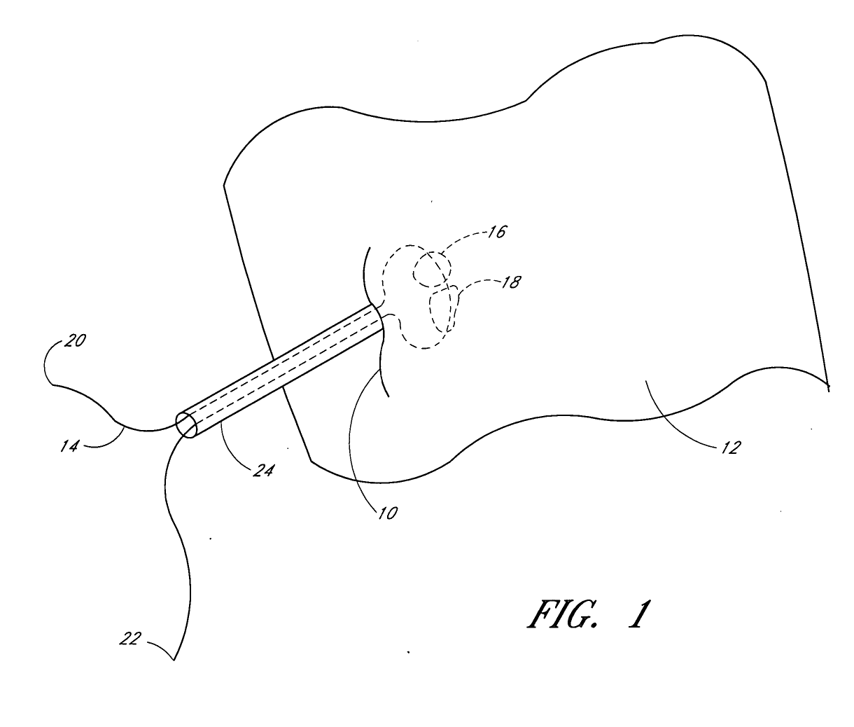

FIG. 1 illustrates the wound site of a patient wherein it may be desired to tie a suture knot. More particularly, FIG. 1 shows an incision 10 in the patient's skin, e.g., a leg 12 of the patient, to perform any sort of treatment on the patient. After the patient has been treated, a suture 14 is introduced into the patient through a catheter sheath introducer (CSI) 24 for the purpose of drawing together tissue portions 16 and 18 (shown in phantom in FIG. 1). Two end portions 20, 22 of the suture 14 extend from the tissue portions 16, 18, respectively, which may, for example, be the result of a wound or an internal incision in a blood vessel or organ. The suture 14 may be introduced into the patient by any suitable manner, including those described in U.S. Pat. No. 5,860,990, U.S. Pat. No. 6,117,144, U.S. Pat. No. 6,562,052, and Applicant's copending application Ser. No. 60/613,636, filed Sep. 27, 2004, all of which are hereby incorporated by reference in their entirety. The suture 14 may be, but is not limited to, 0.007″ diameter biodegradable material or non-biodegradable material, such as polypropylene. The suture 14 may also be braided, or be made of other materials and have other configurations. The suture 14 in FIG. 1 is shown extending from a catheter sheath introducer 24. The suture 14 may also extend directly from an incision in a patient.

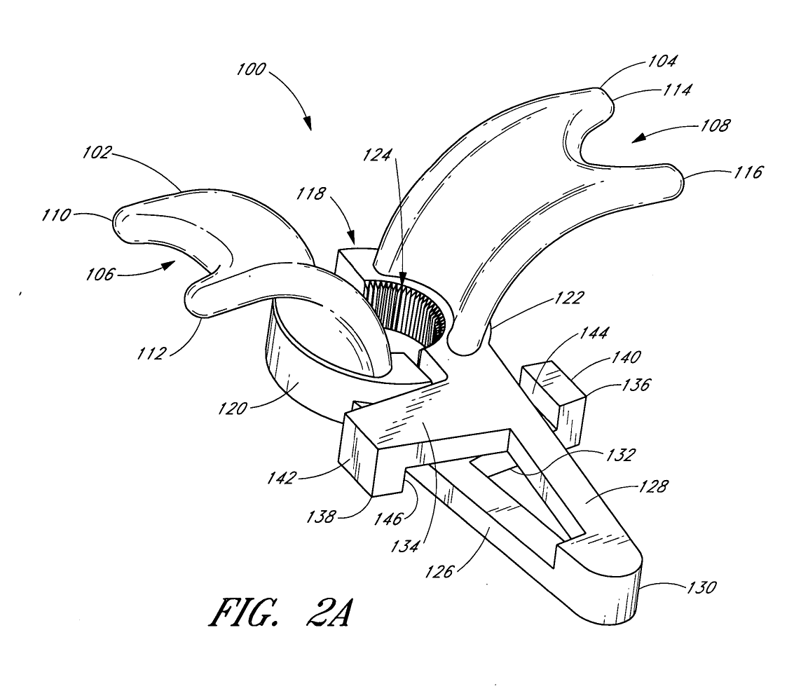

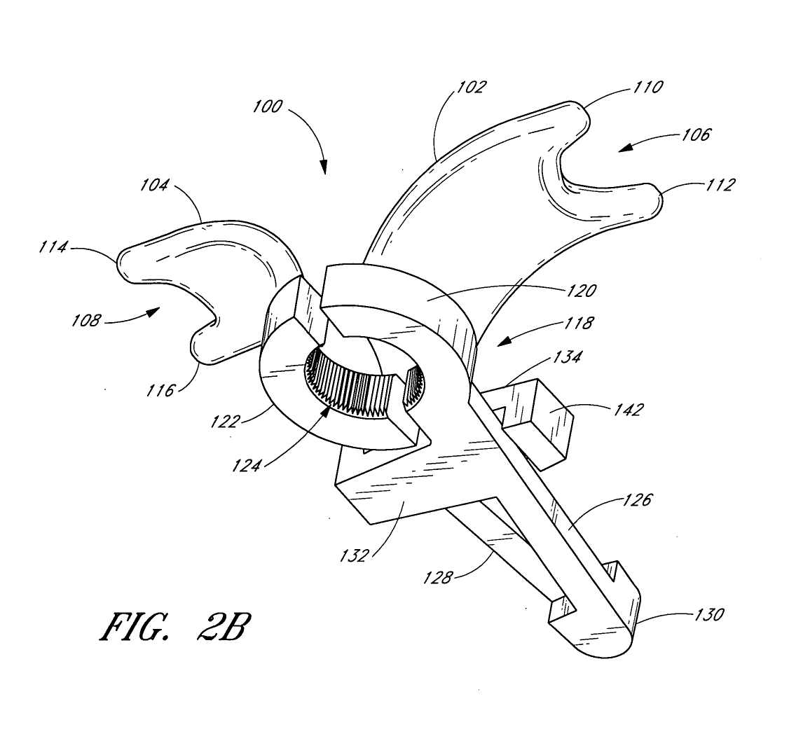

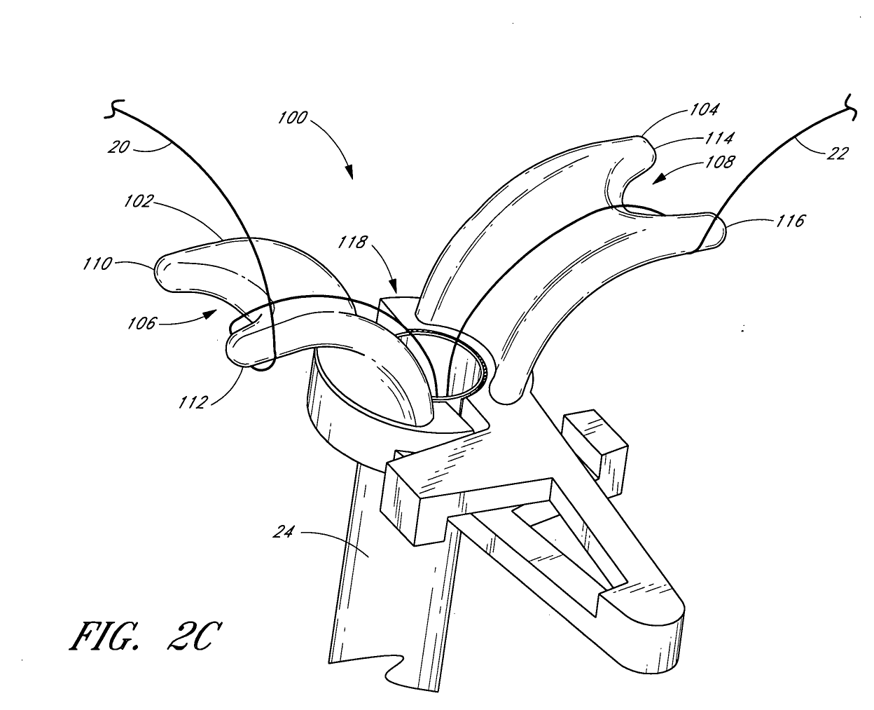

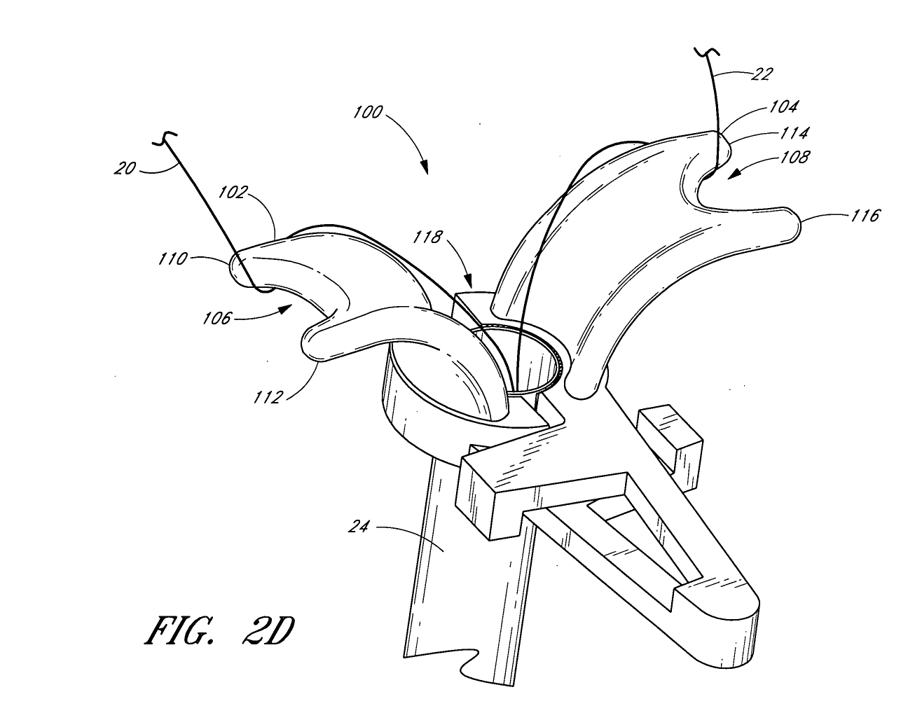

FIGS. 2A and 2B illustrate a first embodiment of a knot-tying assist device 100. The device 100 includes a first receiving arm 102 and a second receiving arm 104, extending away from each other in a generally V shape. At the outermost ends of the arms 102 and 104, the arms include receiving portions 106 and 108 adapted to hold portions of the suture 14. These receiving portions as illustrated are generally U-shaped indentations, defining fingers 110, 112 and 114, 116 on each side of the arms 102 and 104, respectively. At the base 118 of each of the arms 102 and 104, semi-circular engagement portions 120 and 122 are provided, adapted to surround an introducer such as described above. The engagement portions 120 and 122 may have a roughened surface, and in one embodiment, may have ridges 124 to help hold the base against the introducer.

Extending horizontally from each of the semi-circular engagement portions 120 and 122 are bars 126 and 128, which are joined at a pivot point 130. The pivot may comprise a living hinge 130, biased to bring the two semi-circular engagement portions together. Provided along each of the bars 126 and 128 are extension plates 132 and 134, respectively, each of which extends horizontally across the other bar and includes vertical ledges 136 and 138. When the outer surfaces 140 and 142 of the ledges are pressed toward each other, the two semi-circular engagement portions separate to allow the device to be placed over an introducer as described above. The inner surfaces 144 and 146 of the ledges prevent the semi-circular engagement portions from separating too far by providing a stop for each bar 126, 128, and preserve the hinge 130.

The semi-circular engagement portions 120, 122 preferably form arcs having a radius corresponding to the outer radius of an introducer, and may be in the range, for example of about 0.5 to 1 cm. The arms, when the device is positioned around the introducer, preferably separate suture portions a sufficient distance to facilitate a surgeon tying a suture knot. In one embodiment, this distance may be at least 2 cm, more preferably between about 3 to 6 cm, more preferably about 5 cm.

In use, as shown in FIGS. 2C and 2D, the two semi-circular engagement portions are positioned as described above to surround an outer wall of an introducer 24 about its upper or proximal end. The two suture portions 20 and 22 extend out of the introducer and through the base 118 of the device 100. Each of the suture portions may extend over the arms 102 and 104 and down through the indentations 106, 108, and then wrap around fingers 112, 116, as shown in FIG. 2C. Alternatively, the suture portions may pass underneath a first finger 110, 114 of the arms 102, 104, and extend upward through the indentations 106, 108, as shown in FIG. 2D. In either case, the arms desirably separate the two suture portions from each other, making the suture portions easier to access and tie. The suture portions can then be pulled upward, each portion wrapping around a finger on the corresponding arm, and a knot may be tied above the arms. In one preferred embodiment, an improved clinch knot is tied. Once the knot is tied, the suture portions can be released from the device 100, the device can be removed from the introducer 24, and the knot can be cinched to the treatment location, as described below.

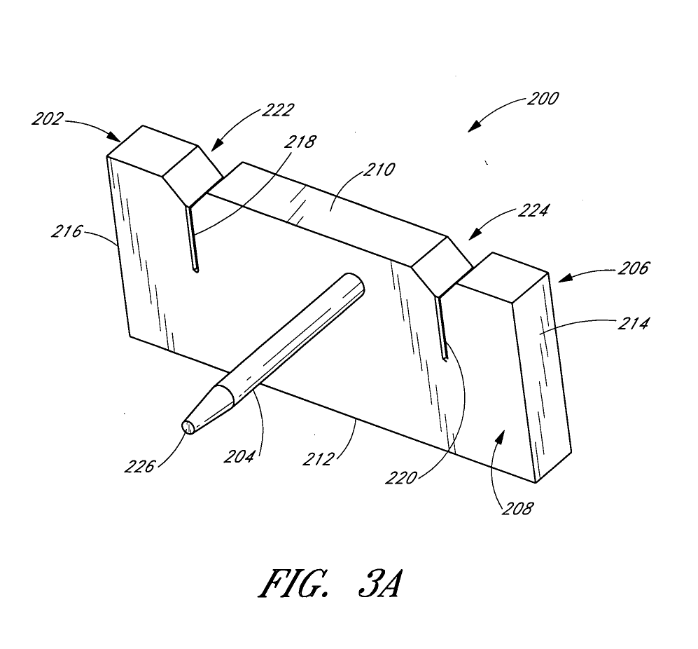

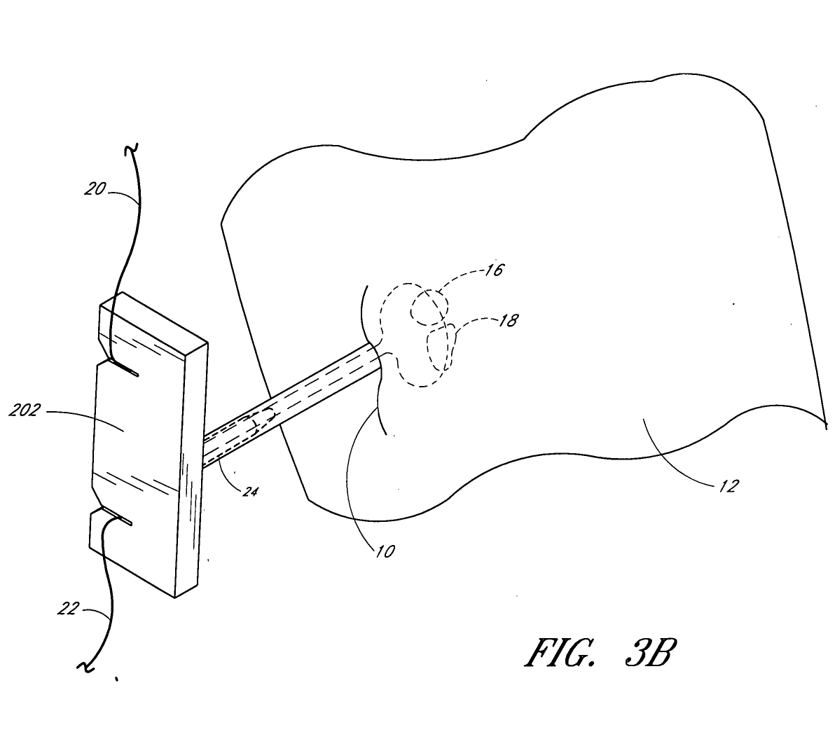

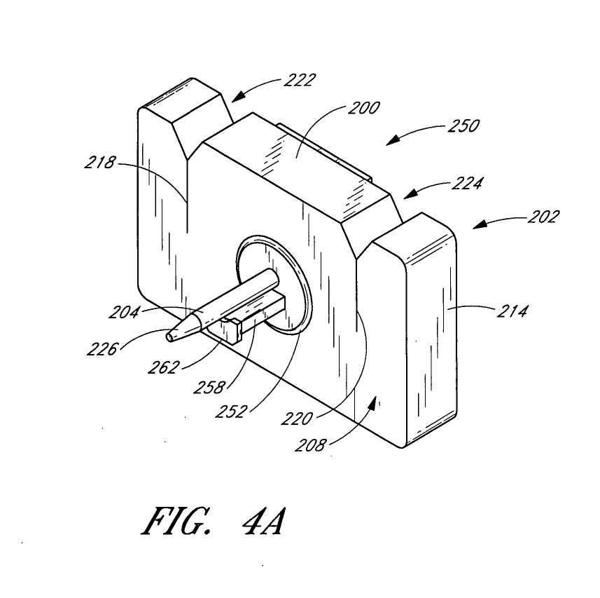

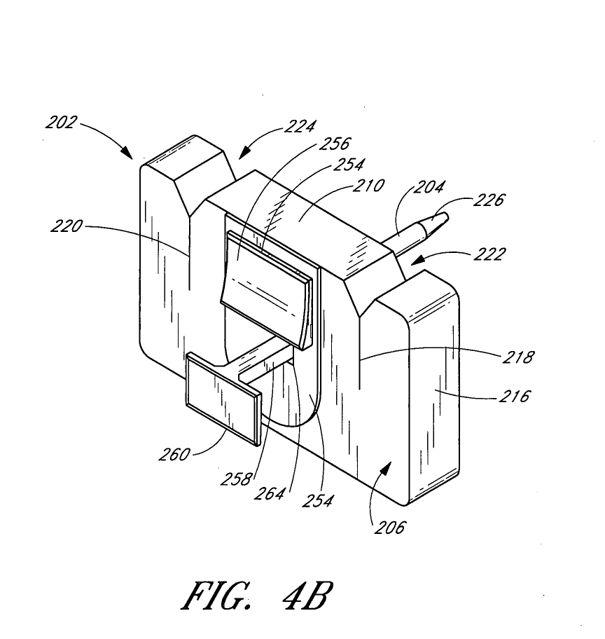

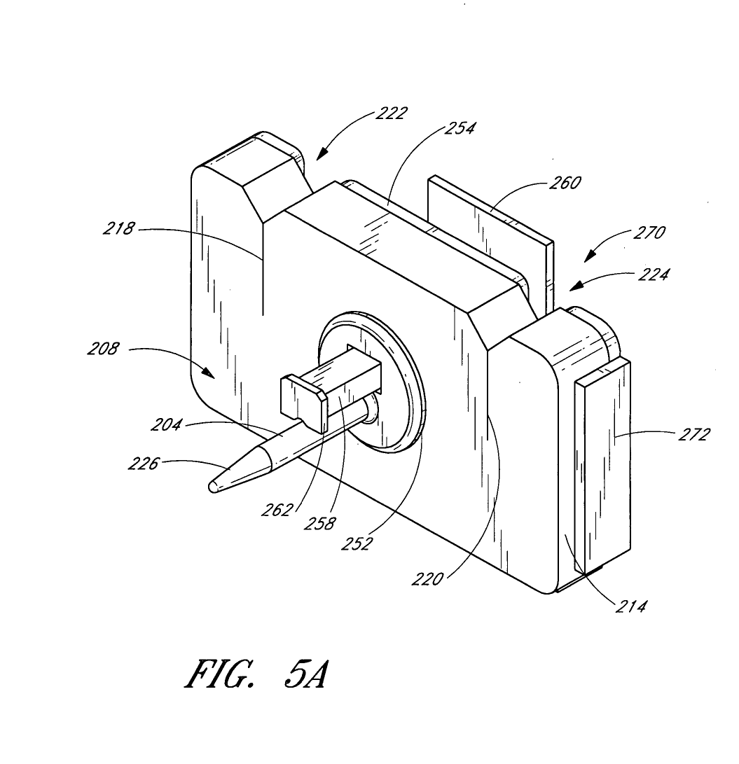

FIG. 3A illustrates a second embodiment of a knot-tying assist device 200 that includes a resilient block 202 and a post 204. The resilient block as illustrated is a rectangular block having an upper surface 206 and a lower surface 208, a front edge 210 and a back edge 212, and side edges 214 and 216. The block has a first slit 218 and a second slit 220 extending into the block from the front edge 210 which are adapted to receive a portion of a suture therein. The slits may extend into the block and may be substantially parallel to each other and to the side edges. At the outer or forward end of each slit, there may be V-shaped notches 222 and 224 to facilitate insertion of a suture portion into the slit. The resilient block may be made of any suitable material, preferably an elastomeric material, for example silicone or rubber, that allows opposite sides of the slit to be separated in order to insert suture portions into the slits. The material is sufficiently elastic to close the slit in order to grip the suture portions there within.

The block as illustrated is preferably rectangular, and has a thickness sufficient to hold suture portions therein. For example, the thickness of the block may be between 0.5 and 2 cm, more preferably about 1 cm, even more preferably about 0.7 cm. It will be appreciated that the block need not be rectangular, but may have any other suitable shape. Additionally, the receiving portions may take the form of slits or any other suitable configuration within the block sufficient to hold and grip the suture portions.

The receiving portions may be provided at any desired location within the block to hold the suture portions a sufficient distance apart. In the illustrated embodiment, the distance between the slits may be sufficient to separate suture portions to enable a surgeon to easily tie a knot with two ends of the suture, and in one embodiment, the slits are positioned at least 2 cm apart, more preferably about 2 to 4 cm apart, more preferably about 3 cm apart. The block may have a length of between about 3 to 6 cm, more preferably about 5 cm, and a width between about 1½ to 3 cm, more preferably about 2 cm. The slits 218 and 220 as illustrated extend parallel to the width of the block, preferably less than half the distance of the width, more preferably about 1 cm or less.

The post 204 extends from the lower surface of the block 202, and includes a tapered or sharpened end 226. The post is preferably made of a rigid material such as hard plastic or metal, and is preferably sized to be inserted into a lumen of an introducer, or directly into an incision in a patient. The post may be connected to the block simply by inserting the post through a hole in the block. FIG. 3B illustrates the embodiment with the post inserted into the lumen of the introducer 24. The post may have a length of about 1 to 3 cm, more preferably about 2 cm. In use, as the post is inserted into the lumen of the introducer, the suture ends 20 and 22 may be held relatively taut by the surgeon or operator. Inserting the post into the introducer then causes the two portions of the suture to be pressed outwardly against the inner wall of the introducer, thereby creating a slight tension on the suture that can be used to hold the tissue or other opening at the treatment location closed. Then, each of the suture portions can be inserted through the V-shaped notches into the slits 218 and 220, while holding tension on each of the suture portions. The engagement between opposite sides of the slits and the suture holds the suture portions in place while separating the ends. The surgeon thus advantageously no longer needs an assistant to hold tension on the suture ends while tying a knot. Accordingly, a knot can be tied with the two suture ends, while the suture portions are held in the block 204. After the knot is tied, the suture portions may be removed from the block, the post can be removed from the introducer, and the knot may be cinched to the treatment location.

FIGS. 4A and 4B illustrate another embodiment of a knot-tying assist device 250, this embodiment including a release or ejection mechanism to help release the device from an introducer or an incision. The post 204 preferably extends from a rigid base 252 provided within a hole in the block 202. On the upper surface 206 of the block, a rigid plate 254 may be integrally formed or connected with the base 252, the plate extending over a portion of the upper surface. The plate 254 may be adhered to the block, or may be connected simply by the base being inserted into the hole within the block. The plate 254 preferably extends to a front edge of the block, between the two slits 218 and 220. The plate may have a raised portion 256 for placement of a thumb. This portion 256 may be contoured, such as with grooves or texturing, to increase its friction coefficient and provide a better grip. A plunger 258 having an enlarged head 260 and an enlarged foot 262 preferably extends through a channel 264 in the plate 254 and the base 252 directly adjacent to the post 204. The plunger is illustrated as being rearward of the post, but can be in any suitable location relative to the post. The plunger is preferably freely moveable relative to the block 202.

The material used for the plate 254, the post 204, the base 252, and the plunger 258 may be any suitable rigid material, including, but not limited to, plastics such as polycarbonate or metal.

In use, when the post 204 is inserted into an introducer or into an incision, the foot 262 of the plunger 258 engages against the upper end of the introducer or skin of a patient, and moves upward as the post is inserted into the introducer or incision. To release the device 250 from the introducer or a patient, the plunger 258 is pressed relatively downward, while the block is pulled relatively upward, causing the post to move relatively upward as the head 260 of the plunger moves toward the block to remove the post from the introducer or a patient. The enlarged foot 262 of the plunger is oversized relative to the channel 264, so that the plunger cannot be removed from the channel.

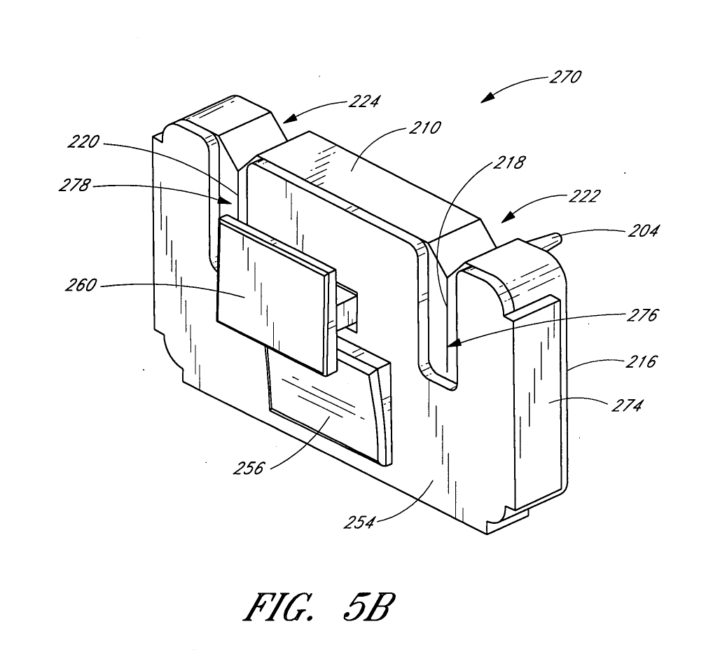

FIGS. 5A and 5B illustrate an embodiment similar to FIGS. 4A and 4B above, and thus, similar reference numerals will be used. In this embodiment, the device 270 includes a plate 254 that extends over a majority of the upper surface of the block 202, and even has side plates 272 and 274 extending over side surfaces 214 and 216 of the block 202. The side plates may be integral with or separate from the plate that covers the upper surface of the block. The plate on the upper surface preferably has gaps 276 and 278 to expose the area where the slits 218 and 220 are located. By the plate being provided on opposite sides of each of the slits, the plate prevents the block from tearing at the slits, and also helps to keep opposite sides of the slits together to ensure that suture portions provided therein are securely held. The plunger in this embodiment is provided forward of the post 204, toward the front edge of the block.

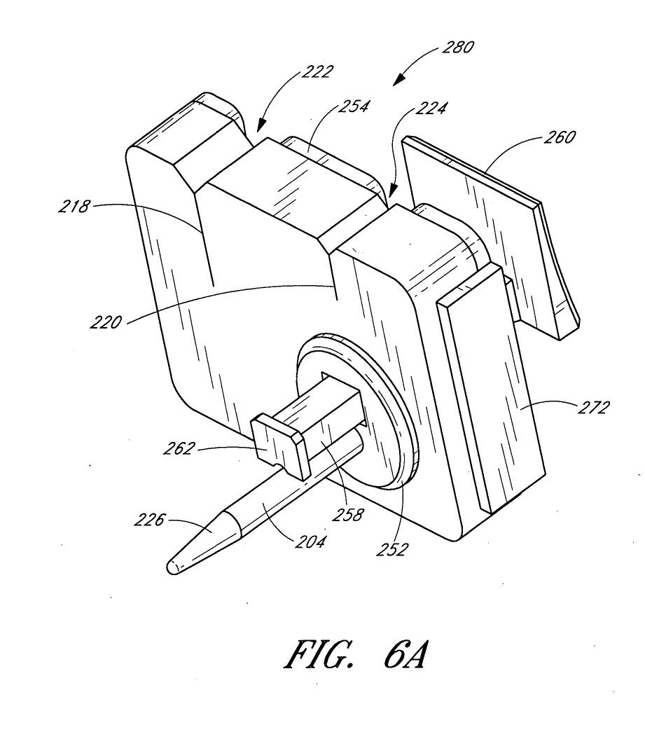

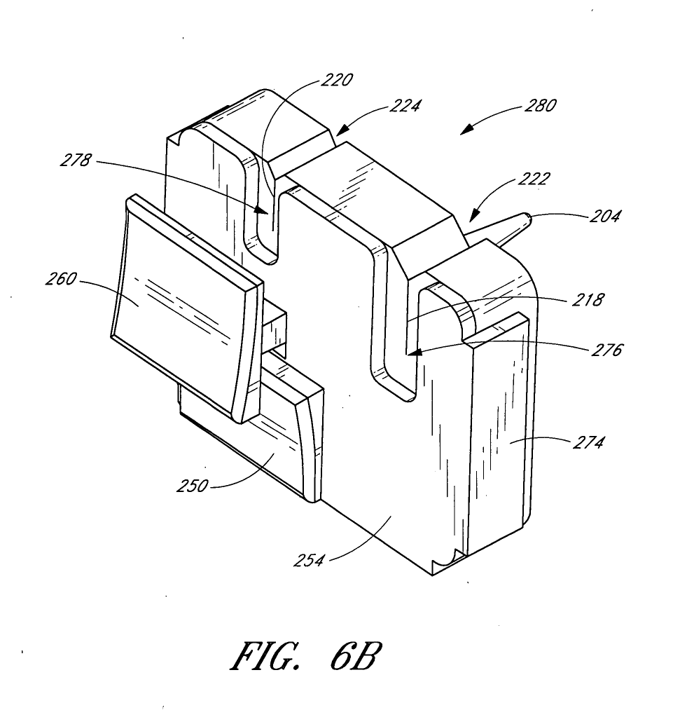

FIGS. 6A and 6B illustrate an embodiment of a knot-tying assist device 280 that has an asymmetrical configuration. One of the slits 220 is provided along the same plane as the post 204, while the other slit 218 is offset from the post by a desired distance. For example, this distance may be about 1 to 2 cm. The slit aligned with the post may be relatively shorter than the offset slit. This asymmetrical configuration may be advantageous because when the suture portions are held in the respective slits, one of the suture portions may be held relatively closer to the introducer or the incision, and thus, be generally aligned with the axis of the introducer. This alignment provides increased stability when tying of the knot, and also facilitates sliding of the knot to the treatment location.

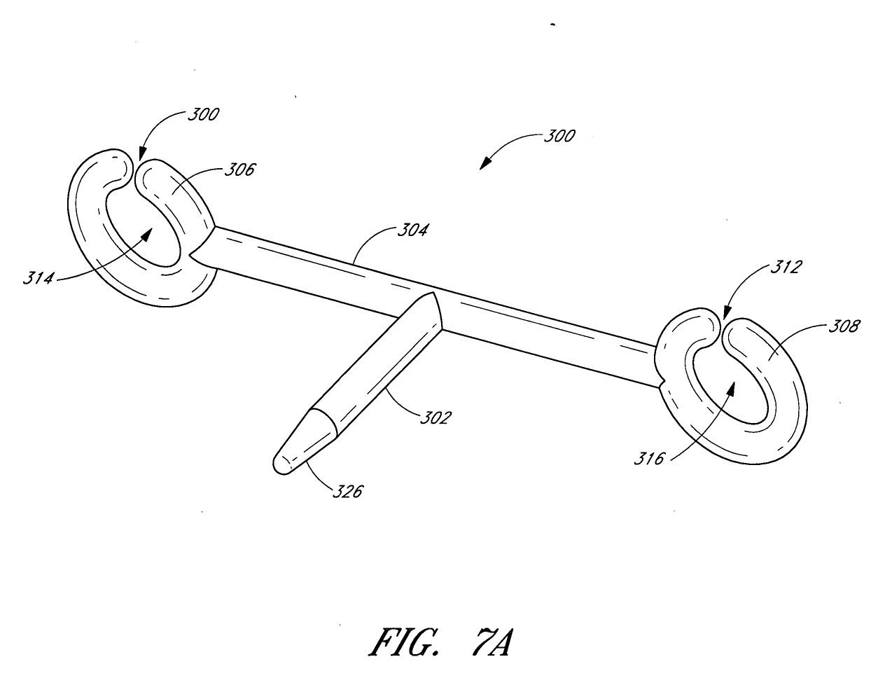

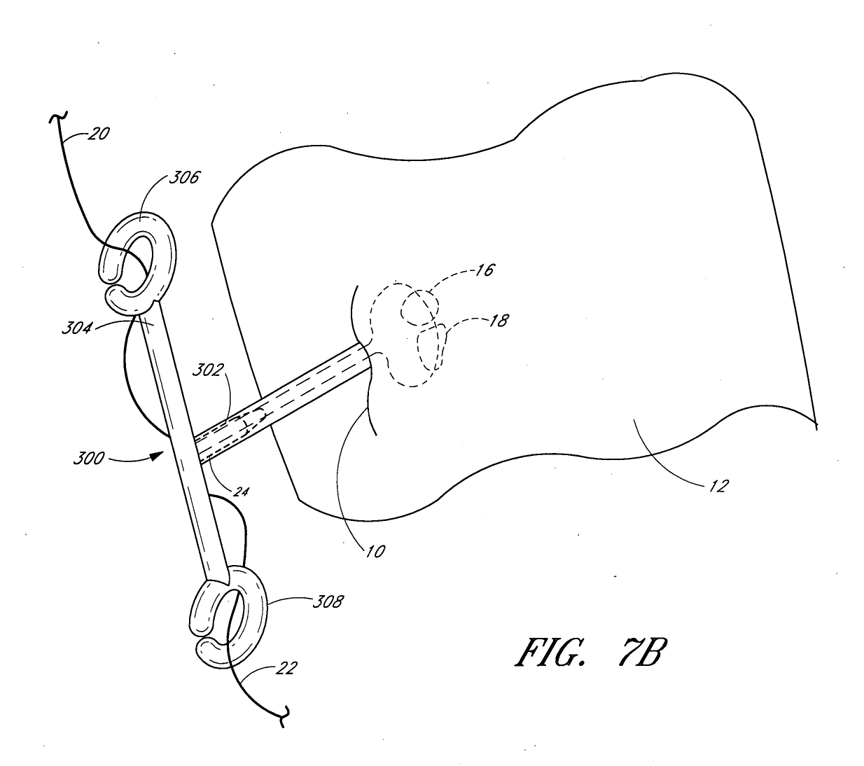

FIG. 7A illustrates another embodiment of a knot-tying assist device 300. The device includes a post 302, and an arm 304, wherein at each end, two generally circular receiving portions 306, 308 are adapted to receive suture portions. The receiving portions 306, 308 are preferably positioned a sufficient distance apart to separate suture portions to facilitate suture tying, and more preferably are at least 2 cm apart, more preferably between about 4 to 6 cm apart, more preferably about 5 cm apart. These circular receiving portions are preferably parallel to the arm 304 and perpendicular to the post. The device 300 is preferably symmetrical such that each receiving portion is the same distance from the post, though asymmetrical designs are also contemplated wherein one of the receiving portions is closer to the post than the other. The receiving portions are preferably donut-shaped and include gaps 310, 312 to allow suture portions to be inserted into the holes or openings 314, 316 defined by the receiving portions. The post and the arm may be integrally formed from a rigid material, for example, a hard plastic such as polycarbonate or metal. As with the embodiments above, the post 302 may have a tapered or sharpened end 326.

FIG. 7B illustrates the device 300 inserted into an introducer 24. As with the embodiments described above, the post 302 may be inserted into the introducer while holding tension on the suture portions, such that the post presses the suture portions against the inner wall of the introducer to hold tension in the suture portions. Each suture portion may then be inserted through the gaps 310 and 312 into the receiving portions 306 and 308 where they are loosely held. These receiving portions separate the suture portions from one another to facilitate tying of a knot while being held within the receiving portions. Once the knot is tied, the suture portions may be released from the device 300, the device 300 may be removed from the introducer, and the knot may be cinched.

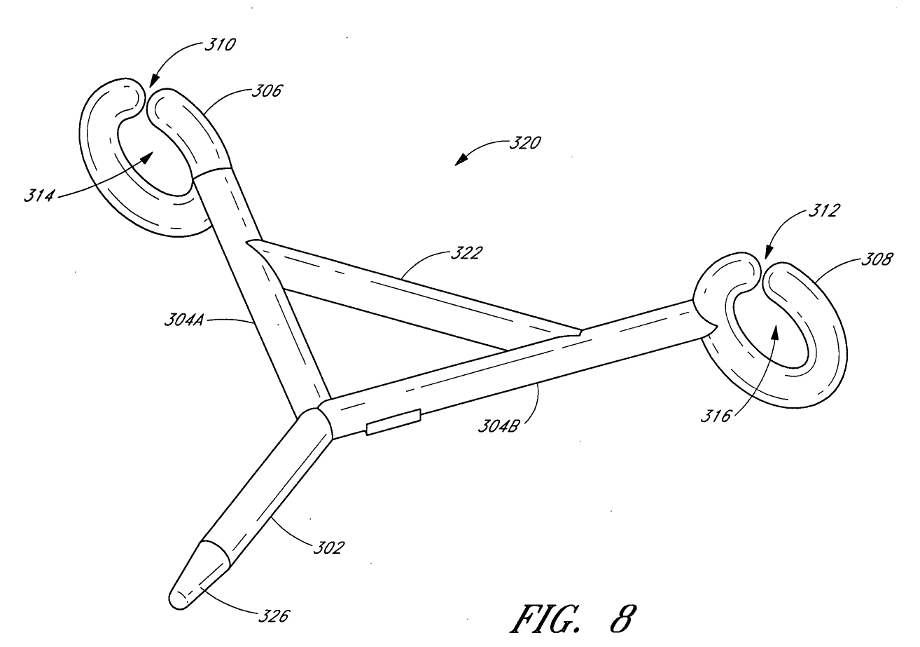

FIG. 8 illustrates another embodiment of a knot-tying assist device similar to that of FIG. 7A, but having a Y-shaped configuration. Arms 304A and 304B extend at an angle away from the post 302, ending at receiving portions 306 and 308 such as described above. Like the embodiment of FIG. 7A, the receiving portions are generally circular and perpendicular to the post. A cross-bar 322 is also provided for support.

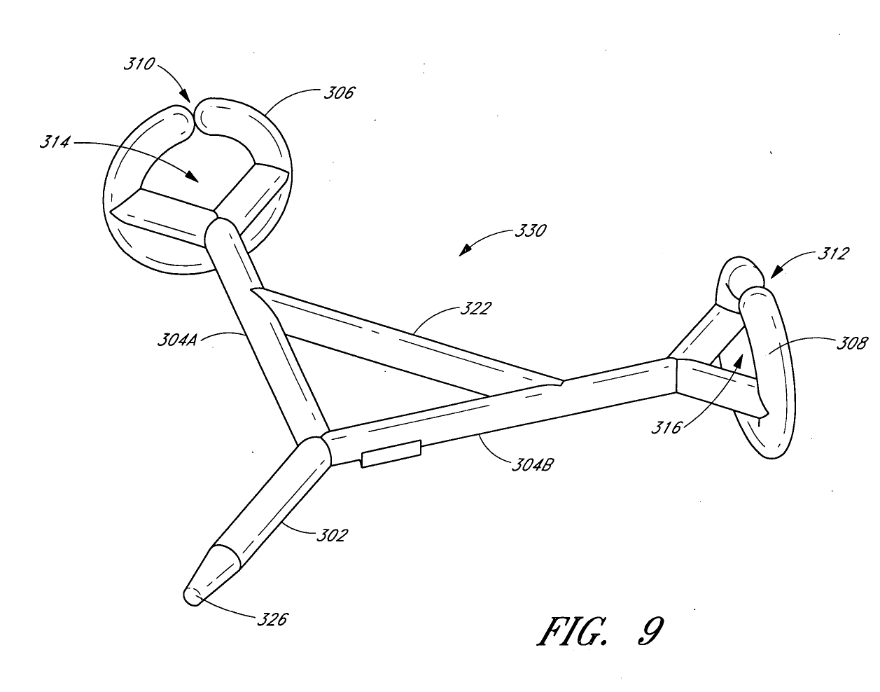

The device 330 of FIG. 9 is similar to the embodiment of FIG. 8, except that the donut-shaped receiving portions 306, 308, instead of being perpendicular to the post, are perpendicular to each of the arms 304A and 304B.

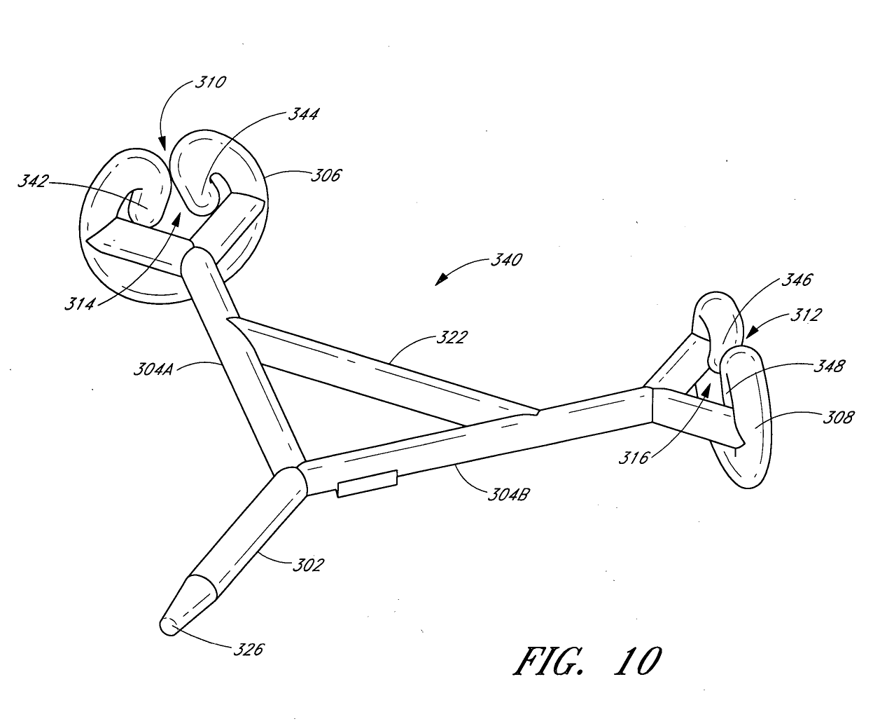

FIG. 10 illustrates a device 340 like that of FIG. 9, except that the circular receiving portions 306, 308 each include ends 342, 344 and 346, 348, respectively, that turn toward each other and then away. This essentially forms a V-shaped portion like those described above which facilitate the suture portions being inserted into the receiving portions. A slight gap 310, 312 may be provided between ends 342, 344, and 346, 348, or these ends may contact each other, but still allow a suture to be passed there through.

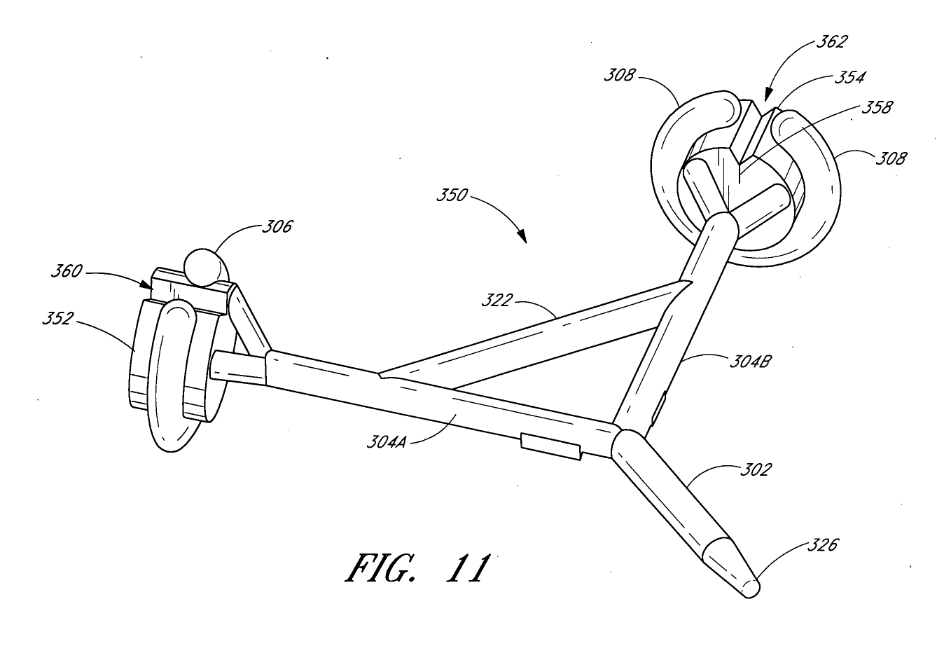

FIG. 11 illustrates another embodiment of a knot-tying assist device 350 having a post 302 and arms 304A and 304B as described above. The receiving portions 306, 308 form generally circular frames, which as illustrated, are perpendicular to arms 304A and 304B. In addition, resilient blocks 352, 354 are provided in the receiving portions 306, 308, which may be made of a suitable material such as silicone or rubber. These blocks are preferably circular or cylindrical in shape, and are adapted to be held in the circular frame provided at the ends of the arms. Each of the blocks preferably comprises a slit 356, 358 like in the resilient blocks described above, and also includes V-shaped notches 360 and 362. Thus, suture portions may be placed in the slits and be fixedly held by opposite sides of the slits.

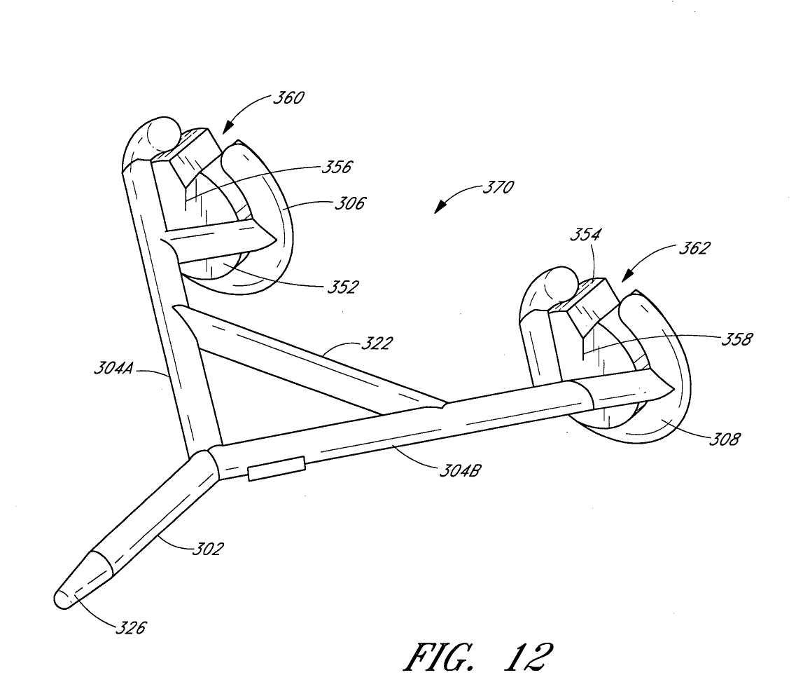

FIG. 12 illustrates a device 370 similar to the embodiment of FIG. 11, except that the circular frames 306, 308 holding the resilient blocks 352, 354, are generally perpendicular to the post 304.

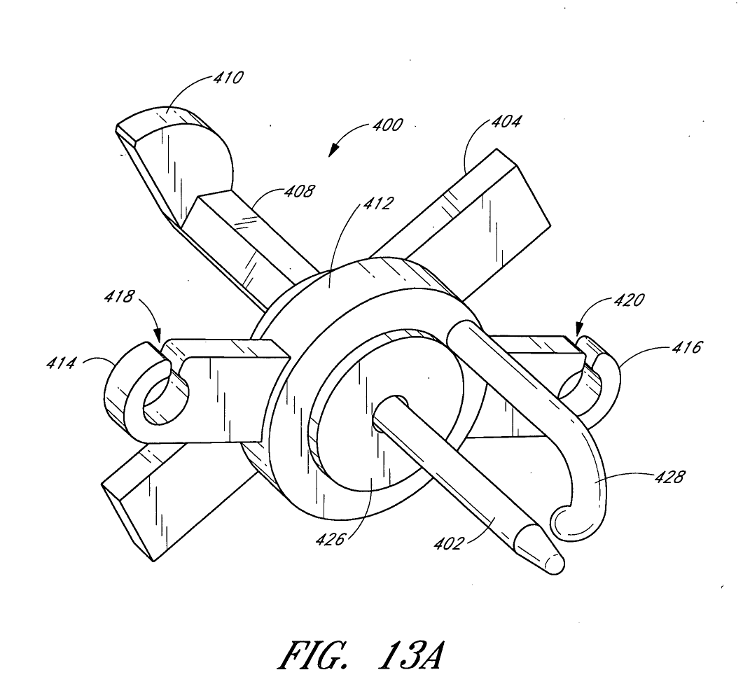

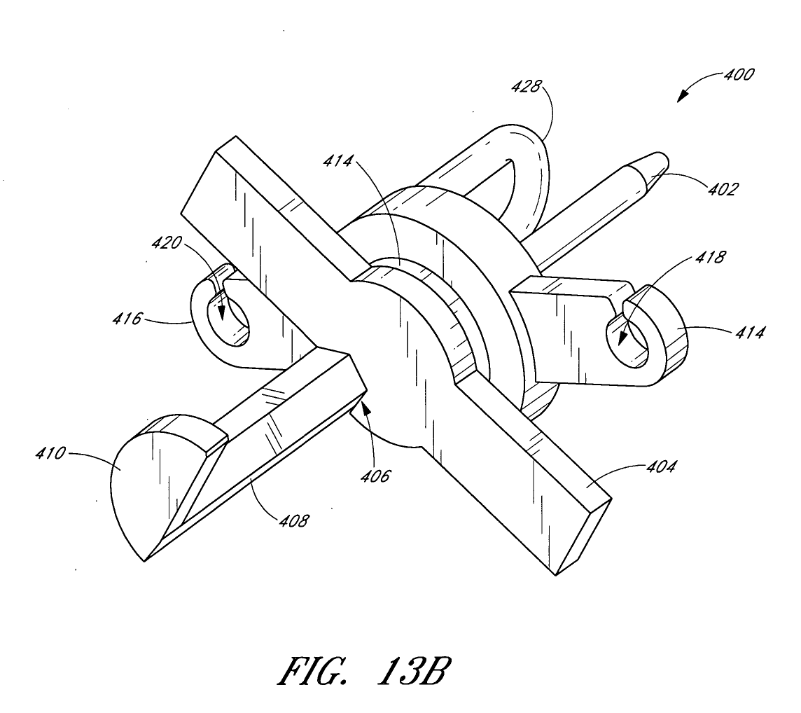

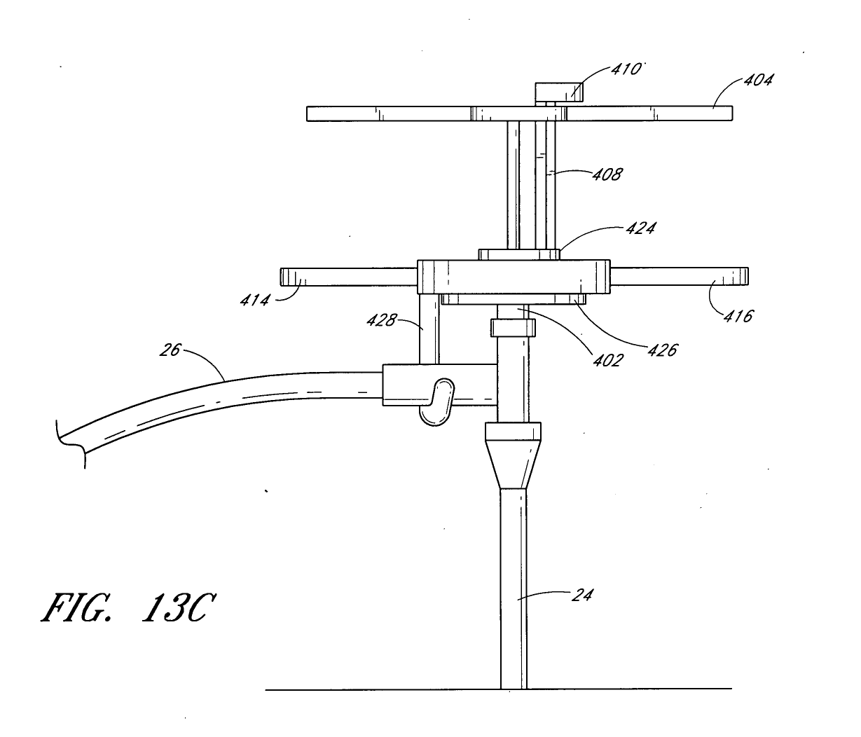

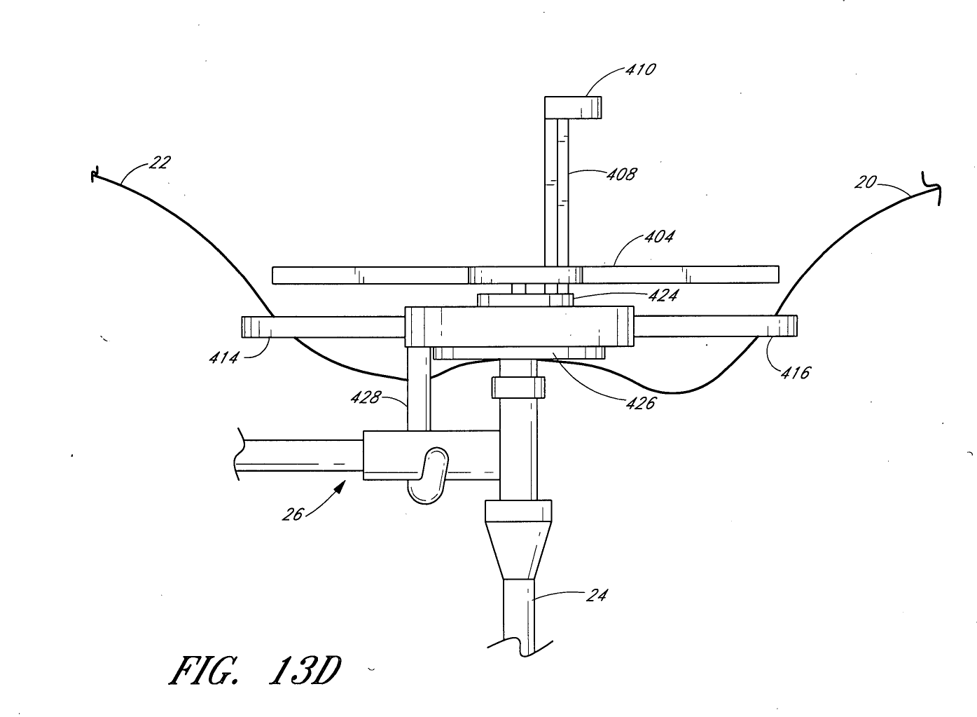

FIGS. 13A and 13B illustrate another embodiment of a knot-tying assist device 400. In this embodiment, a post 402 is provided with is rigidly connected or integrally formed with a plate 404, forming a T-shape. The plate includes a notch 406 in a central portion of the plate which forms a channel along which a plunger 408 can slide, the plunger having an enlarged head 410. The plunger 408 and the post 402 are slideable axially relative to one another, but are rotationally fixed due to the notch. The plunger 408 is connected to a base 412 having arms 414 and 416 extending outwardly therefrom, each arm having a suture receiving portion 418, 420. The suture receiving portions may loosely or firmly hold suture portions using embodiments such as described above. A hole (not shown) is provided through the base 412 to allow the post 402 to slide relatively there through.

The plunger 408 is fixed axially relative to the base by an upper plate 424 rigidly connected or integrally formed with the plunger provided on an upper surface of the base, and a lower plate 426 rigidly connected or integrally formed with the plunger provided on the lower surface of the base. These plates each have a hole through which the post 402 extends, thereby holding the plunger 408 and the base 412 relatively together. However, these plates are not adhered to the base, and therefore, they may rotate relative to the base about the post 402. A J-shaped hook 428 is rigidly connected to or integrally formed with the base 412 and extends downward from the base.

In use, the hook 428 is provided to hook around a connector that may be provided at the end of an introducer. As shown in FIG. 13C, the hook 428 may hook underneath a line 26 extending from the side of a proximal end of an introducer 24. The hook desirably fixes rotationally the arms 414, 416 with respect to the introducer, while the bottom plate 426 of the plunger engages the top end of the introducer. With the hook in place, the post 402 may be pushed down into the introducer, relative to the plunger 408, using the plate 404. Before pushing down the post 402, the plate 404 may be rotated relative to the arms 414, 416 so that the plate 404 is at an angle with respect to the arms 414, 416 and doesn't interfere with the suture receiving portions 418 and 420. With the post pressed into the introducer, as shown in FIG. 13D, the suture portions 20 and 22 may extend through the receiving portions 418, 420 in the arms 416, 418. These receiving portions may loosely hold the suture portions, or may securely hold the suture portions, as described above. After a knot is tied, the suture portions may be released from the receiving portions 418, 420, and the operator may press down on the plunger 408, while pulling up on the plate 404, to remove the post 402 from the introducer.

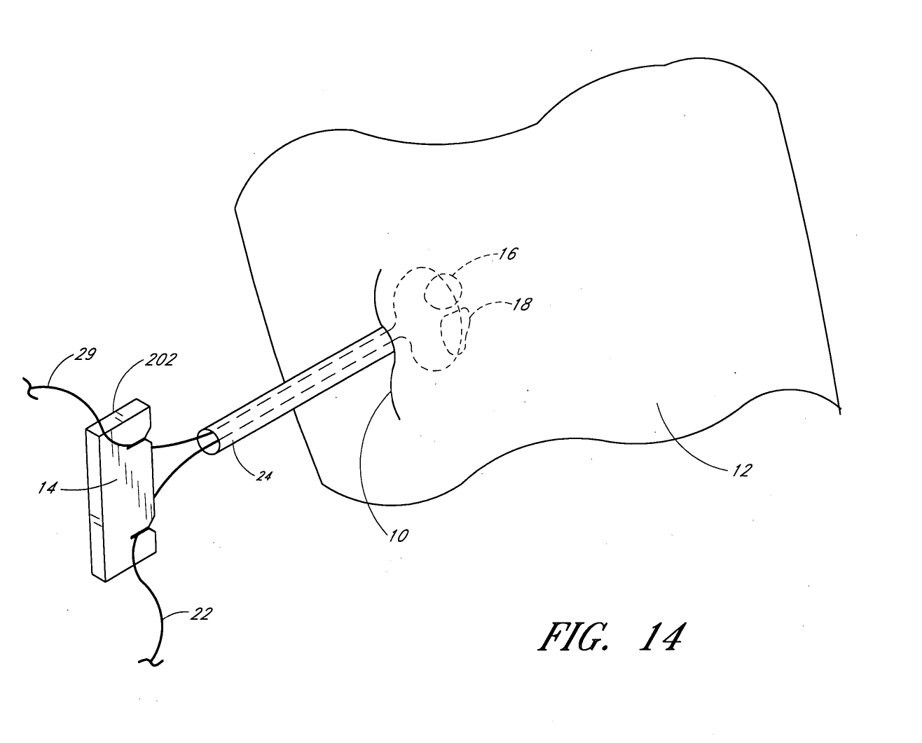

It will be appreciated that other embodiments similar to those described above are contemplated. In a simpler embodiment, as shown in FIG. 14, a resilient block 202 by itself may be used without a post to separate suture portions. The block may be suspended by the suture portions while a surgeon ties a knot. After the knot is tied, the block may be removed and the knot may be cinched. Other embodiments contemplated include having more than two suture receiving portions, such as when multiple suture portions or ends are to be tied.

One preferred knot that may be tied while separating and/or holding the suture end portions using a device such as described above is an improved clinch knot. Other types of knots may also be tied. After the knot has been tied and the knot-tying assist device has been released from the suture portions, the knot is desirably cinched as is known to one of ordinary skill in the art. In some embodiments, only one of the suture portions is released from the device, while the other portion is held in the device while the knot is cinched. A knot cinching or pushing device may also be used, such as described in Applicant's application entitled “Knot Pusher,” Ser. No. 09/571,759, filed May 15, 2000, which is hereby incorporated by reference in its entirety.

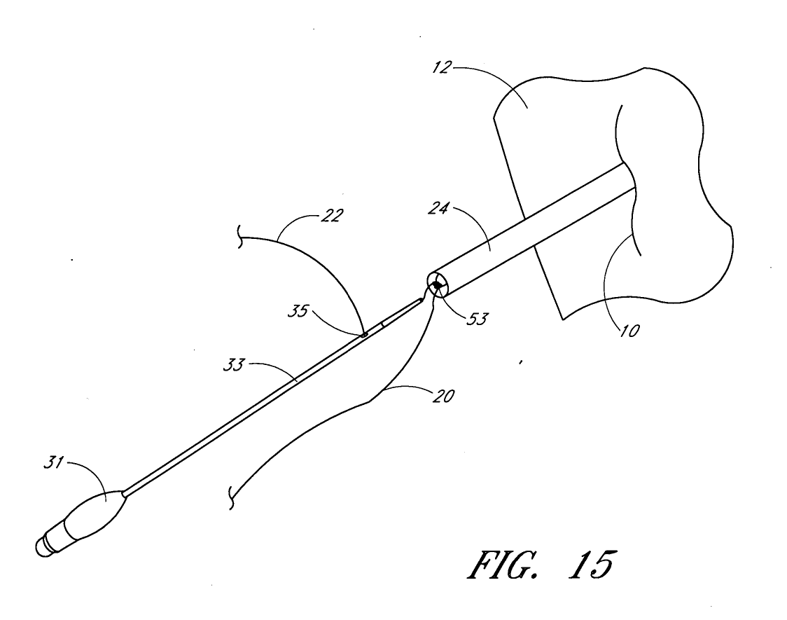

FIG. 15 illustrates a knot 53 after the suture portions have been removed from one of the devices as described above. One of the suture end portions 22 extends through a knot cinching device 27 having a holder 35, described in the application incorporated by reference above. The suture end portion 22 extends through a lumen at the distal end of the device 27, and exits through an opening 35. A practitioner cinches the knot 53 by first holding the suture end 22 taut in one hand while simultaneously holding the CSI 24 with the same hand. With the other hand, the practitioner then pushes the knot 53 with the tubular member 33 of the cinching device 27 down the CSI 24 and towards the internal incision. Because the inside diameter of the lumen of the cinching device 27 is substantially smaller than the diameter of the knot 53, the knot 53 does not enter into the lumen. Once the knot 53 is positioned just proximal to the internal incision, the practitioner may then proceed to cut back the suture 14 so that the suture ends 20, 22 do not extend out of the incision 10 in the patient's skin. A suture cutter may be used for this purpose.

The embodiments of the present invention advantageously separate suture portions making them easier to tie, while also providing tension on suture portions due to a holding force either in a suture receiving portion or by virtue of a post engaging the suture within an introducer or an incision. In addition, where mechanisms are provided to engage the device with an introducer, such mechanisms stabilize the position of the device, and therefore the suture, relative to the introducer. This prevents twisting of the suture ends.

It should be understood that certain variations and modifications of this invention will suggest themselves to one of ordinary skill in the art. The scope of the present invention is not to be limited by the illustrations or the foregoing descriptions thereof.

What is claimed is:

1. A knot-tying assist device for facilitating tying a knot in two suture portions extending from a treatment location, comprising:
a first receiving portion configured to receive a first suture portion;
a second receiving portion configured to receive a second suture portion; and
an engagement portion configured to secure the knot-tying assist device relative to the treatment location;
wherein said first and second receiving portions are spaced apart on said knot-tying assist device.

2. The knot-tying assist device of claim 1, wherein said first and second receiving portions are spaced apart between about 2-6 cm.

3. The knot-tying assist device of claim 1, wherein said first and second receiving portions are configured to loosely hold said first and second suture portions.

4. The knot-tying assist device of claim 1, wherein said first and second receiving portions comprise first and second slits.

5. The knot-tying assist device of claim 1, wherein said first and second receiving portions comprise first and second holes.

6. The knot-tying assist device of claim 1, wherein said first and second receiving portions comprise resilient material having slits for receiving said suture portions.

7. The knot-tying assist device of claim 1, wherein said engagement portion comprises a post extending transversely to an axis defined between the first and second receiving portions.

8. The knot-tying assist device of claim 7, wherein said post is sized to be inserted into an introducer positioned in an incision at the treatment location.

9. The knot-tying assist device of claim 8, further comprising an introducer, wherein the post is sized to be inserted into the introducer.

10. The knot-tying assist device of claim 8, further comprising an ejector mechanism for releasing the knot-tying assist device from said introducer.

11. A knot-tying assist device for facilitating tying a knot in two suture portions extending from a treatment location, comprising:
a post having a first and second end;
at least one arm connected to the second end of the post;
a first receiving portion configured to receive a first suture portion, said first receiving portion being circular and including a gap to allow said first suture portion to be inserted into an opening defined by said first receiving portion;
a second receiving portion configured to receive a second suture portion, said second receiving portion being circular and including a gap to allow said second suture portion to be inserted into an opening defined by said second receiving portion;
wherein said first receiving portion is located at a first end of said at least one arm and said second receiving portion is located at a second end of said at least one arm.

12. The knot-tying assist device of claim 11, wherein said first and second receiving portions are spaced apart between about 2-6 cm.

13. The knot-tying assist device of claim 11, wherein said at least one arm comprises first and second portions that extend in a T-shaped configuration from the second end of said post.

14. The knot-tying assist device of claim 11, wherein said at least one arm comprises first and second arms extending in a Y-shaped configuration from the second end of said post.

15. The knot-tying assist device of claim 11, wherein said first and second circular portions further comprise a resilient material positioned in said first and second openings with a slit configured to hold and apply tension to said first and second suture portions.

16. The knot-tying assist device of claim 11, wherein the first end of the post is tapered.

17. A method of tying two suture ends extending from a treatment location, comprising:
holding each of two suture portions in a device that separates the two suture portions a desired distance from each other;
tying a knot in the two suture portions while the two suture portions are held; and
removing the two suture portions from the device.

18. The method of claim 17, wherein the device holds the two suture portions in tension.

19. The method of claim 17, wherein the two suture portions extend from the treatment location through a lumen of an introducer sheath, and the device comprises a post, wherein the method further comprises inserting the post into the lumen of the introducer.

20. The method of claim 17, further comprising cinching the knot down to the treatment location.
